(19) United States
(12) Patent Application Publication
(10) Pub. No.: US 2015/0196281 A1
(43) Pub. Date: Jul. 16, 2015

(54) MEDICAL DATA PROCESSING DEVICE, MEDICAL DATA PROCESSING METHOD, AND ULTRASOUND DIAGNOSTIC DEVICE

(71) Applicants: KONICA MINOLTA, INC., Chiyoda-ku, Tokyo (JP); PANASONIC CORPORATION, Osaka (JP)

(72) Inventors: Kazuya Takagi, Machida-shi (JP); Mitsuyoshi Takiguchi, Sapporo-shi (JP); Kensuke Nakamura, Sapporo-shi (JP)

(73) Assignee: KONICA MINOLTA, INC., Chiyoda-ku, Tokyo (JP)

(21) Appl. No.: 14/419,930

(22) PCT Filed: Aug. 2, 2013
(86) PCT No.: PCT/JP2013/004697

§ 371 Date: Feb. 5, 2015

(30) Foreign Application Priority Data
Aug. 7, 2012 (JP) 2012-174648

(51) Int. Cl. A61B 8/08; A61B 8/00; A61B 5/00

(52) U.S. Cl. CPC A61B 8/5223; A61B 5/7264; A61B 8/481; A61B 8/085; A61B 8/461; A61B 8/4444

(57) ABSTRACT

A medical data processing device classifies a tumor type by using a first numerical sequence indicating a time series variation of a feature value of a tumor region including a tumor. The first numerical sequence is obtained from echo signals obtained from a living organism after administration of a contrast agent. The medical data processing device includes a first classifier that extracts, from the first numerical sequence, a first numerical sequence portion of a classification interval having a predefined time period shorter than the entire time of the first numerical sequence and classifies the tumor type by using the first numerical sequence portion.

### TECHNICAL FIELD

The present invention is related to medical data processing devices, medical data processing methods, and ultrasound diagnostic devices, and particularly to a medical data processing device that classifies a tumor type by using information obtained from echo signals obtained from a living organism after administration of a contrast agent.

### BACKGROUND ART

Contrast-enhanced ultrasound is one diagnostic imaging method by which blood vessels can be imaged with high sensitivity, by administration of a contrast agent into the blood vessels. The primary component of such a contrast agent is bubbles having strong ultrasound wave reflectivity.

In cancer diagnosis, after a screening test for a tumor that is suspected to be a cancer, a differential diagnosis is performed to establish whether the tumor is a cancer. Contrast-enhanced ultrasound is currently being used in cancer diagnosis, particularly in differential diagnosis.

The contrast agent is administered in a bolus. Typically, the contrast agent arrives at the tumor after a period of time, and increases intensity in an ultrasound image. In other words, contrast-enhancement is established. FIG. 1 illustrates an example of a time intensity curve (TIC) that plots a change over time of intensity in an ultrasound image. In a diagnostic classification of tumor type, an observer observes the contrast-enhancement in the ultrasound image and classifies whether the tumor is benign or malignant (cancer).

Currently, such tumor type classification is based on subjectivity of the observer. Therefore, there is a problem that diagnosis relies on the observer. Thus, several objective diagnostic methods have been proposed.

Patent Literature 1 discloses a method for associating a fitting coefficient with a tumor type, in which a TIC is fitted by a predefined modeling function.

Further, Patent Literature 2 discloses a method for classifying tumor type by performing pattern matching between a TIC and a representative pattern for each tumor type.

### CITATION LIST

### Patent Literature

Patent Literature 1: Japanese Patent No. 4706003
Patent Literature 2: Japanese Patent Application Publication No. 2010-005263
Patent Literature 3: U.S. Pat. No. 5,632,277
Patent Literature 4: U.S. Pat. No. 5,706,819
Patent Literature 5: U.S. Pat. No. 5,577,505

### SUMMARY OF INVENTION

### Technical Problem

In such tumor type classification, an improvement in performance of tumor type classification is desired.

Thus, an aim of the present invention is to provide a medical data processing device that improves performance of tumor type classification.

### Solution to Problem

The medical data processing device pertaining to an aspect of the present invention is a medical data processing device that classifies a tumor type by using a first numerical sequence indicating a time series variation of a feature value of a tumor region including a tumor, the first numerical sequence being obtained from echo signals obtained from a living organism after administration of a contrast agent, the medical data processing device comprising: a first classifier that extracts, from the first numerical sequence, a first numerical sequence portion of a classification interval having a predefined time period shorter than the entire time period of the first numerical sequence and classifies the tumor type by using the first numerical sequence portion.

Note that these general or specific aspects may be implemented as any one of a system, a method, an integrated circuit, a computer program, and a computer-readable storage medium such as a CD-ROM, or as any combination of a system, a method, an integrated circuit, a computer program, and a storage medium.

### Advantageous Effects of Invention

The present invention provides a medical data processing device that improves performance of tumor type classification.

### BRIEF DESCRIPTION OF DRAWINGS

FIG. 1 illustrates an example of a TIC.

FIG. 2 is a block diagram of an ultrasound diagnostic device pertaining to embodiment 1.

FIG. 3 is a block diagram of a TIC generator pertaining to embodiment 1.

FIG. 4 is a block diagram of a type classifier pertaining to embodiment 1. FIG. 5 is a diagram illustrating an example of time intervals pertaining to embodiment 1.

FIG. 6 is a flowchart of an ultrasound image generation process pertaining to embodiment 1.

FIG. 7 is a flowchart of a TIC generation process pertaining to embodiment 1.

FIG. 8 is a diagram illustrating an example of a display screen pertaining to embodiment 1.

FIG. 9 is a flowchart of a TIC normalization process pertaining to embodiment 1.

FIG. 10A is a diagram illustrating an example of a tumor perfusion start time pertaining to embodiment 1.

FIG. 10B is a diagram illustrating an example of a parenchymal perfusion start time pertaining to embodiment 1.

FIG. 11 is a flowchart of a tumor type classification process pertaining to embodiment 1.

FIG. 12 is a diagram illustrating an example of thresholds pertaining to embodiment 1.

FIG. 13 is a diagram illustrating an example of a table indicating tumor types pertaining to embodiment 1.

FIG. 14 is a diagram for describing an example of the tumor type classification process pertaining to embodiment 1.

FIG. 15 is a diagram for describing an example of the tumor type classification process pertaining to embodiment 1.

FIG. 16A is a diagram illustrating an example of tumor type classification results pertaining to embodiment 1, displayed as bars.

FIG. 16B is a diagram illustrating another example of a tumor type classification result pertaining to embodiment 1, displayed as a bar.

FIG. 16C is a diagram illustrating an example of tumor type classification results pertaining to embodiment 1, displayed as marks.

FIG. 17 is a diagram illustrating an example display of time intervals having a high contribution to the tumor type classification pertaining to embodiment 1.

FIG. 18 is a block diagram of a medical data processing device pertaining to embodiment 1.

FIG. 19 is a flowchart of a tumor type classification process pertaining to embodiment 2.

### EMBODIMENTS

(Knowledge Foundation of Present Invention)

The inventors discovered the following problem related to the techniques described under the heading “Background Art”.

Both Patent Literature 1 and Patent Literature 2 disclose tumor type classification based on TICs and are objective diagnostic techniques.

However, poor matches are common when fitting modeling functions and data, and therefore selecting a modeling function is difficult. Further, a fitting coefficient is normally selected such that error between the fitting coefficient and the data is minimized and matching is normally performed such that similarity between the data and a standard pattern is maximized. When considering that data includes information that is useful in tumor type classification and information that is not useful in tumor type classification, the information that is useful may be buried in the data by such methods.

The medical data processing device pertaining to an aspect of the present invention is a medical data processing device that classifies a tumor type by using a first numerical sequence indicating a time series variation of a feature value of a tumor region including a tumor, the first numerical sequence being obtained from echo signals obtained from a living organism after administration of a contrast agent, the medical data processing device comprising: a first classifier that extracts, from the first numerical sequence, a first numerical sequence portion of a classification interval having a predefined time period shorter than the entire time period of the first numerical sequence and classifies the tumor type by using the first numerical sequence portion.

In this way, the medical data processing device extracts and uses information useful for type classification and therefore improves performance of type classification.

For example, the classification interval may be provided in a plurality, the first classifier may classify the tumor type by using the first numerical sequence portion of each of the classification intervals and output a plurality of corresponding intermediate results, which each indicate a result of the first classifier classifying the tumor type, and the medical data processing device may further comprise a second classifier that classifies the tumor type by using the intermediate results and contribution ratios pre-associated with the classification intervals on a one-to-one basis.

In this way, the medical data processing device performs type classification using the plurality of classification intervals, and therefore improves performance of type classification.

For example, the second classifier, for each classification interval, may calculate a multiplication result by multiplying the intermediate result of the classification interval by the contribution ratio pre-associated with the classification interval, calculate a multiplication sum by summing all the multiplication results, and classify the tumor type based on the multiplication sum.

For example, the feature value may be a difference between intensity of the tumor region and a parenchymal region that does not include the tumor.

In this way, the medical data processing device performs type classification by using the difference between intensity of the tumor region and another region useful in type classification, and therefore further improves classification performance. In particular, when the tumor type is a malignant tumor, perfusion into circulation out of the tumor region is said to be faster than in said another region (parenchymal region), and therefore using the difference between intensities is useful for type classification.

For example, the first classifier may acquire a second numerical sequence indicating a time series variation of a feature value of a parenchymal region that does not include the tumor and determine a perfusion start time of the contrast agent from the second numerical sequence, and the classification interval may be a time interval predefined by using the perfusion start time as a reference time.

In this way, the medical data processing device takes into account a perfusion time difference between the tumor region and said another region useful in type classification, and therefore further improves classification performance.

For example, the first classifier may classify the tumor type based on which is greater out of an average value of the first numerical sequence portion included in the classification interval and a preset threshold.

In this way, the medical data processing device efficiently classifies tumor type from the first numerical sequence portion of the classification interval.

For example, the first classifier may classify the tumor type based on which is greater out of a time change value of the first numerical sequence portion included in the classification interval and a preset threshold.

In this way, the medical data processing device efficiently classifies tumor type from the first numerical sequence portion of the classification interval.

For example, the medical data processing device may further comprise a display that displays the tumor type as classified by the first classifier.

In this way, an observer can check a classified tumor type in situ.

For example, the first classifier may determine probabilities of the tumor being each of a plurality of types, and the display may display the plurality of types and the probabilities of the tumor being each of the types.

In this way, an observer can check the tumor type classified and the probability of the tumor type. Further, the observer can check probabilities of the tumor being other tumor types.

For example, the display may display the probabilities as a graphic.

In this way, an observer can intuitively check a classified tumor type.

For example, the display may emphasize a highest probability type among the plurality of types.

In this way, an observer can check a tumor type having a high probability more easily.

For example, the medical data processing device may further comprise a display that displays the first numerical sequence as a graph and displays, associated with the graph of the first numerical sequence, the classification intervals and the contribution ratios corresponding to the classification intervals.

In this way, an observer can check time series variation of information obtained from the living organism indicated by the first numerical sequence at the same time as checking the classification interval and contribution ratio used in classifying the tumor type in the first numerical sequence.

For example, the medical data processing device may further comprise an input device that receives a change to the contribution ratios made by an operator, wherein the second classifier re-classifies the tumor type based on the change made to the contribution ratios.

In this way, an observer can perform type classification while adjusting a contribution ratio based on subjectivity and experience of the observer.

Further, a medical data processing method pertaining to an aspect of the present invention is a medical data processing method of classifying a tumor type by using a first numerical sequence indicating a time series variation of a feature value of a tumor region including a tumor, the first numerical sequence being obtained from echo signals obtained from a living organism after administration of a contrast agent, the medical data processing method comprising: a first classifying step of extracting, from the first numerical sequence, a first numerical sequence portion of a classification interval having a predefined time period shorter than the entire time of the first numerical sequence and classifies the tumor type by using the first numerical sequence portion.

In this way, the medical data processing method extracts and uses information useful for type classification and therefore improves performance of type classification.

Further, an ultrasound diagnostic device pertaining to an aspect of the present invention is an ultrasound diagnostic device, comprising: an ultrasound probe that acquires echo signals from a living organism after administration of a contrast agent; a numerical sequence generator that generates, from the echo signals, a first numerical sequence that indicates a time series variation of a feature value of a tumor region including a tumor; and the medical data processing device of any one of claims 1-13 that classifies tumor type by using the first numerical sequence.

In this way, the ultrasound diagnostic device extracts and uses information useful for type classification and therefore improves performance of type classification.

Note that these general or specific aspects may be implemented as any one of a system, a method, an integrated circuit, a computer program, and a computer-readable storage medium such as a CD-ROM, or as any combination of a system, a method, an integrated circuit, a computer program, and a storage medium.

The following describes embodiments of the present invention with reference to the drawings. Identical elements are assigned the same symbols, and description thereof may be omitted.

In the following, type is used as a word indicating whether a tumor is benign or malignant and as a word indicating a classification of tumor (for example, in the case of liver cancer, hepatocellular carcinoma, cholangiocellular carcinoma, undifferentiated carcinoma, biliary cystadenocarcinoma, carcinoid tumor, etc.)

Note that the embodiments described below indicate specific examples of the present invention. The values, shapes, materials, elements, positions and connection modes of elements, steps, order of steps, etc., indicated in the following embodiments are merely examples and are not intended to limit the present invention. Further, among the elements in the following embodiments, elements that are not recited in independent claims indicating the highest level concept are described as optional elements.

### Embodiment 1

An ultrasound diagnostic device 100 pertaining to the present embodiment, for each of a plurality of classification intervals, generates an intermediate result by classifying a type of tumor by using TIC information included in the corresponding classification interval, multiplies each intermediate result by a predefined contribution ratio, sums a plurality of results of such multiplication, and classifies a final tumor type by using a result of such summing. In this way, the ultrasound diagnostic device 100 improves type classification.

The following describes structure and operations of a system of the ultrasound diagnostic device 100.

<Structure>

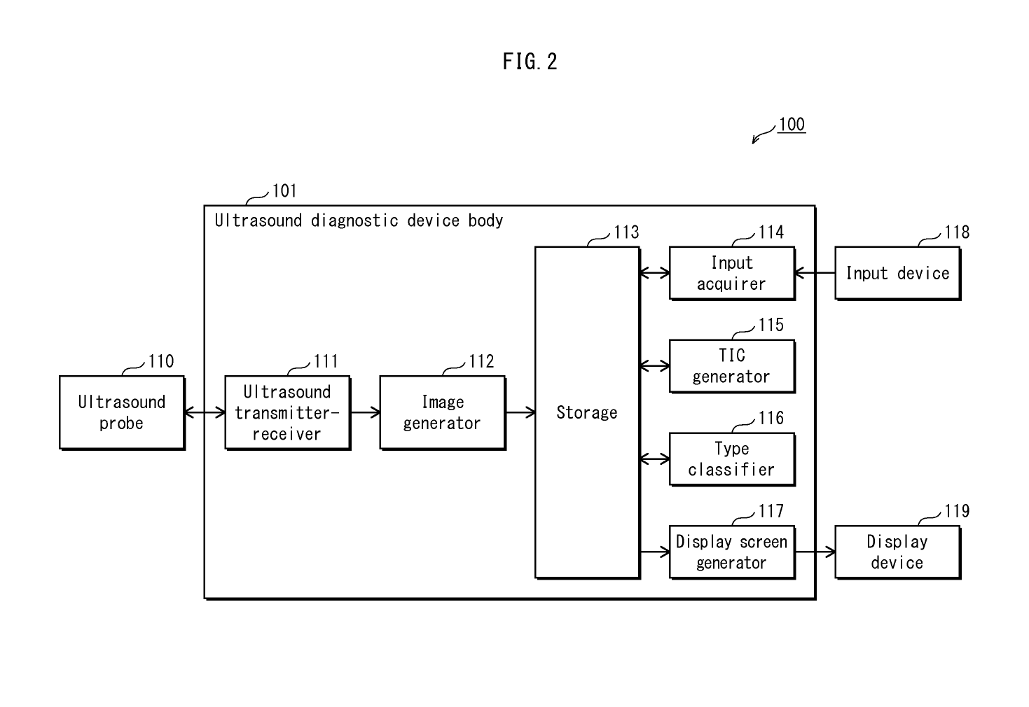

FIG. 2 is a block diagram illustrating structure of the ultrasound diagnostic device 100 pertaining to the present embodiment.

As illustrated in FIG. 2, the ultrasound diagnostic device 100 includes an ultrasound diagnostic device body 101, an ultrasound probe 110, an input device 118, and a display device 119. The ultrasound diagnostic device body 101 includes an ultrasound transmitter-receiver 111, an image generator 112, a storage 113, an input acquirer 114, a TIC generator 115, a type classifier 116, and a display screen generator 117. The ultrasound diagnostic device body 101 is connected by wired or wireless means to the ultrasound probe 110, the input device 118 (track-ball, button, touch panel, etc.), and the display device 119 (display, etc.)

The ultrasound probe 110 converts an electrical signal inputted from the ultrasound transmitter-receiver 111 into an ultrasound wave and transmits the ultrasound wave to a subject. Subsequently, the ultrasound probe 110 acquires an echo signal that is returned by the ultrasound wave being reflected at the subject, converts the echo signal into an electrical signal, and outputs the electrical signal to the ultrasound transmitter-receiver 111.

The ultrasound transmitter-receiver 111 generates the electrical signal that the ultrasound wave is based on, and outputs the generated electrical signal to the ultrasound probe 110. Further, the ultrasound transmitter-receiver 111 converts the electrical signal outputted from the ultrasound probe 110 to a digital echo signal and outputs the digital echo signal to the image generator 112.

The image generator 112 generates an ultrasound image by converting the digital echo signal outputted from the ultrasound transmitter-receiver 111 into intensity values. At such time, as ultrasound images, a fundamental image is formed primarily from fundamental components centered on a transmission frequency and a harmonic image is formed primarily from harmonic components. Subsequently, the image generator 112 stores generated ultrasound images in the storage 113.

In the storage 113, in addition to various images and setting data, machine learning parameters used in the type classification are stored. Note that the storage 113 may be external memory connected by wired or wireless means to the ultrasound diagnostic device body 101.

The input acquirer 114 acquires information indicating a section of interest and regions of interest as specified by an operator via the input device 118, and stores acquired information in the storage 113. Here, the section of interest is a cross-section from a plurality of sections of a time series, and is used for selecting the regions of interest. Further, the regions of interest are regions used to classify a type of tumor, and specifically include a region including a tumor.

The TIC generator 115 reads, from the storage 113, information indicating the section of interest and the regions of interest along with the ultrasound image, and generates TICs of the regions of interest. The TIC generator 115 is one example of a numerical sequence generator that generates a first numerical sequence (TIC) from the echo signals. Details thereof are described later. Subsequently, the TIC generator stores generated TICs in the storage 113.

The type classifier 116 reads the TICs and the machine learning parameters used in type classification from the storage 113, and classifies a type of tumor. Such processing is described later. Subsequently, the type classifier stores a classification result in the storage 113.

The display screen generator 117 reads the ultrasound image from the storage 113, and generates an image for setting the regions of interest and the section of interest. Further, the display screen generator 117 reads the classification result from the storage 113 and generates a display screen indicating the classification result. Subsequently, the display screen generator 117 outputs the display screen to the display device 119 causing the display screen to be displayed.

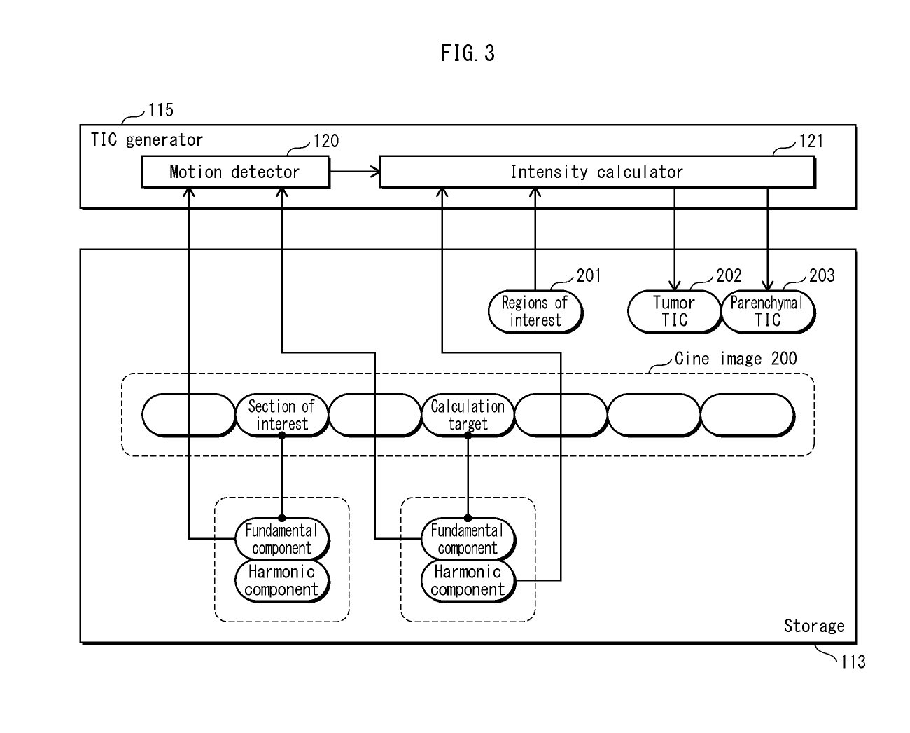

The following is a detailed description of structure of the TIC generator 115. FIG. 3 is a block diagram illustrating structure of the TIC generator 115.

As illustrated in FIG. 3, the TIC generator 115 includes a motion detector 120 and an intensity calculator 121.

Further, a cine image 200 including a plurality of ultrasonic images of a time series and information indicating the regions of interest (regions of interest 201) are stored in the storage 113.

The motion detector 120 reads two fundamental images from the storage 113. One of the two fundamental images serves as a reference for motion detection, for example an image of the section of interest prior to administration of a contrast agent. The other of the two fundamental images is a TIC calculation subject. The motion detector 120 detects a motion vector of the two fundamental images and outputs the detected motion vector to the intensity calculator 121.

The intensity calculator 121 reads information indicating the regions of interest from the storage 113 and corrects a region of interest in the image that is a TIC calculation subject by using the motion vector outputted by the motion detector 120. Subsequently, the intensity calculator 121 reads a harmonic image of the TIC calculation subject from the storage 113 and calculates average intensities of the regions of interest of the harmonic image. Here, the regions of interest includes two regions: a tumor region and a parenchymal region. Here, the tumor region is a region including a tumor and the parenchymal region is a normal region not including a tumor. The intensity calculator 121 calculates an average intensity of the tumor region and an average intensity of the parenchymal region. Further, the intensity calculator 121 performs the above series of processes for each image acquired in a time series. Finally, the intensity calculator 121, for each of the tumor region and the parenchymal region, arranges the average intensities in a time series, and stores the time series as TICs (tumor TIC 202 and parenchymal TIC 203).

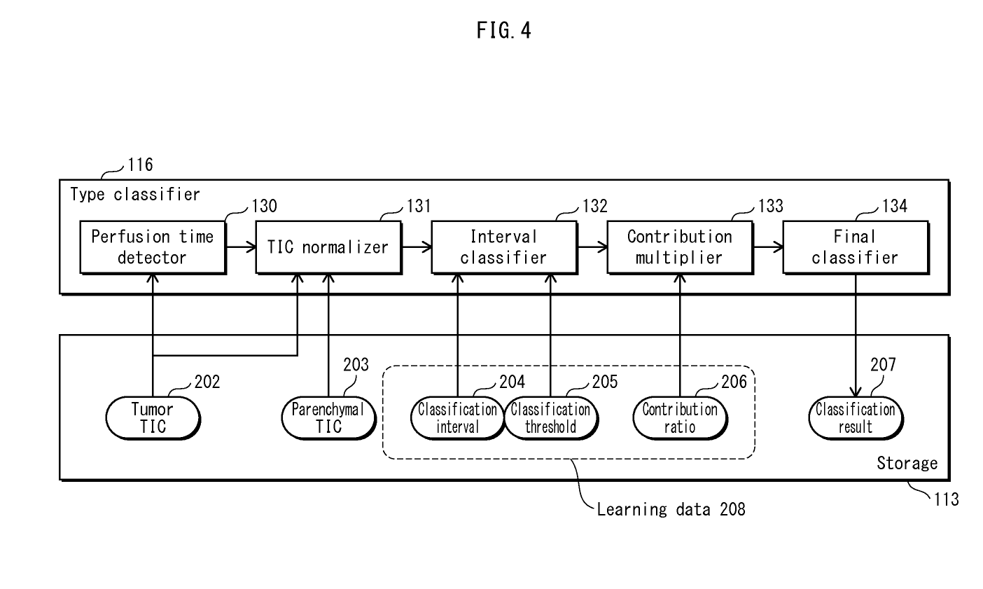

The following is a detailed description of structure of the type classifier 116. FIG. 4 is the block diagram illustrating structure of the TIC generator 115.

As illustrated in FIG. 4, the type classifier 116 includes a perfusion time detector 130, a TIC normalizer 131, an interval classifier 132, a contribution multiplier 133, and a final classifier 134.

The perfusion time detector 130 reads the tumor TIC 202 from the storage 113. Subsequently, the perfusion time detector 130 detects an increase of the tumor TIC 202 as a perfusion start time Subsequently, the perfusion time detector 130 outputs the perfusion start time to the TIC normalizer 131.

The TIC normalizer 131 acquires the perfusion start time outputted by the perfusion time detector 130. Further, the TIC normalizer 131 reads the tumor TIC 202 and the parenchymal TIC 203 from the storage 113 and generates a difference TIC that is the difference between the tumor TIC 202 and the parenchymal TIC 203. Further, the TIC normalizer 131 resets the perfusion start time of the difference TIC as a reference time (for example, time zero). The TIC normalizer 131 outputs the TIC after performing the above processes to the interval classifier 132.

The interval classifier 132 acquires the TIC after normalization outputted by the TIC normalizer and reads information (classification interval 204) indicating a classification interval including machine learning data 208 and information indicating a threshold (classification threshold 205) from the storage 113.

FIG. 5 illustrates an example of time intervals selected by using such machine learning data. An example classification interval is one or more time intervals having a high contribution to the type classification from a time interval group 160 that represents all times of a TIC divided by predefined time intervals.

The contribution ratio is determined from previously-acquired case data by using a predefined machine learning algorithm. For example, machine learning may be performed as follows. First, the previously-acquired case data is divided into an identical time interval group, and type classification is performed only using the data in each interval. Accuracy of matching between the type as classified and a type of the case data is judged. This is performed for many instances of case data. The contribution ratio corresponds to an accuracy rate of each time interval, and one or more time intervals having a high accuracy rate are set as classification intervals.

The interval classifier 132 classifies which feature value (average intensity, variance, gradient, etc.) of a tumor type a feature value within the classification intervals is close to. The classification result is outputted as a numerical value. For example, the interval classifier 132 outputs +1 when a tumor is classified as benign and −1 when a tumor is classified as malignant. The interval classifier 132 performs such classification for all the classification intervals, and outputs the classification results to the contribution multiplier 133.

The contribution multiplier 133 acquires the classification results outputted by the interval classifier 132, and reads contribution ratios 206 included in the machine learning data 208 from the storage 113. The contribution ratios 206 are the same as the contribution ratios mentioned above. The contribution multiplier 133 calculates a sum of products of the intermediate results and the contribution ratios 206 as a type evaluation value, and outputs the type evaluation value to the final classifier 134. In other words, the contribution multiplier 133, for each classification interval, multiplies the intermediate result of the classification interval by the contribution ratio associated with the classification interval in order to generate a plurality of multiplication values, and calculates the type evaluation value by summing the multiplication values.

The final classifier 134 acquires the type evaluation value from the contribution multiplier 133 and classifies the type of the tumor by using the type evaluation value. Subsequently, the final classifier 134 outputs a classification result 207 to the storage 113.

<Operations>

The following describes operation flow of the ultrasound diagnostic device 100 pertaining to the present embodiment.

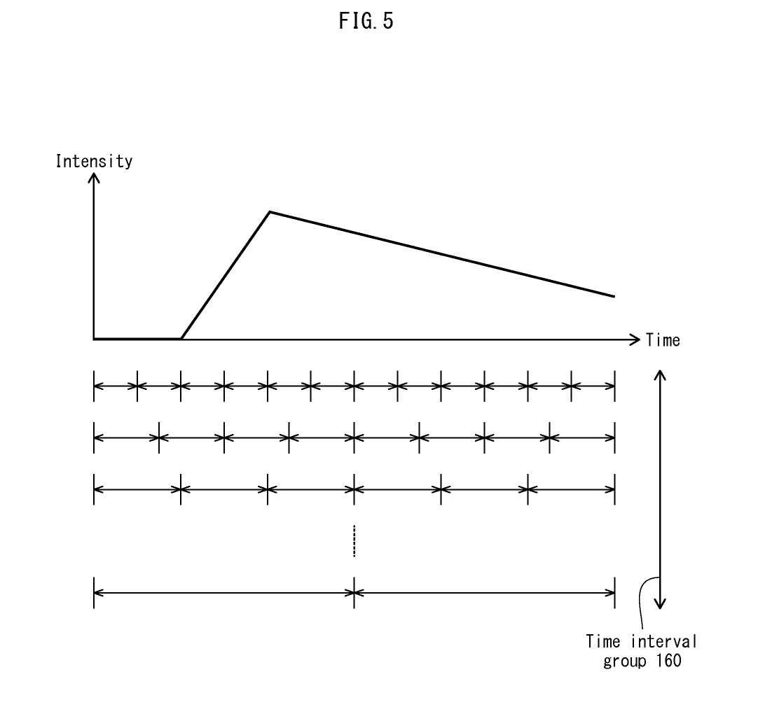

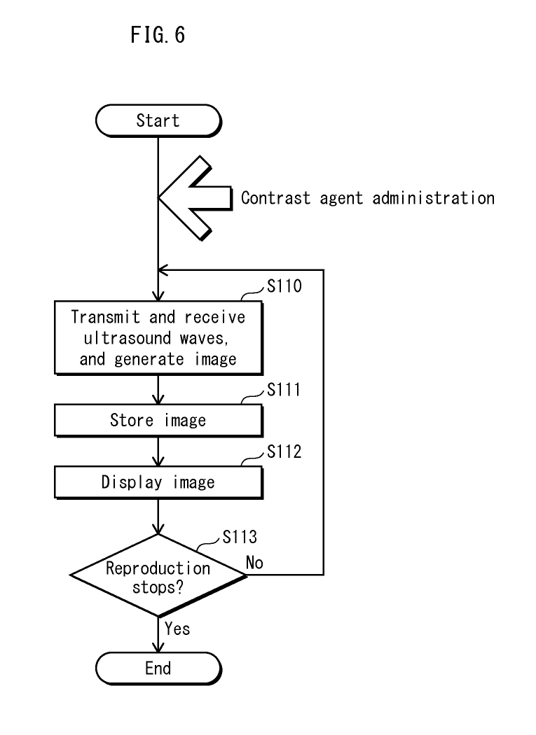

FIG. 6 is a flowchart of an ultrasound image generation process pertaining to the present embodiment.

The following description assumes operation after an operator has administered a contrast agent to the subject. Here, description is of an example in which a tumor is classified into two classes: benign and malignant.

### Step S110

Initially, the ultrasound transmitter-receiver 111 transmits two phase-inverted pulses (for details, see Patent Literature 3-5) in order to extract harmonic components including a lot of the contrast agent. Subsequently, the ultrasound transmitter-receiver 111 generates a summed signal obtained by summing two received echo signals and generates a non-summed signal obtained by not summing the two received echo signals. The ultrasound transmitter-receiver 111 outputs the summed signal to the image generator 112 as a harmonic component echo signal. On the other hand, the ultrasound transmitter-receiver 111 performs a filter process on the non-summed signal to suppress harmonic components and outputs a signal after the filter process to the image generator 112 as a fundamental component echo signal.

Subsequently, the image generator 112 performs quadrature detection on each of the harmonic component echo signal and the fundamental component echo signal outputted by the ultrasound transmitter-receiver 111, converting the echo signals to amplitude values. The image generator 112 fits the amplitude values to the resolution and gradation of the display screen by performing decimation and logarithmic compression on the amplitude values. Further, the image generator 112 generates ultrasound images by performing, on the signals after the above processes, an interpolation process called scan conversion to align scan lines to actual scale. In this way, an ultrasound image is generated for each of the fundamental component echo signal and the harmonic component echo signal.

### Step S111

Subsequently, the image generator 112 stores the fundamental image and the harmonic image on the storage 113. The fundamental image is an ultrasound image generated from the fundamental component echo signal and the harmonic image is an ultrasound image generated from the harmonic component echo signal.

### Step S112

Further, in order that the operator can check the ultrasound images in real time, the display screen generator 117 reads the harmonic image from the storage 113 and generates a display screen including the harmonic image. The display device 119 displays the display screen so generated.

### Step S113

Subsequently, when the operator instructs via the input device 118 that reproduction is to be stopped, the ultrasound transmitter-receiver 111 stops transmission and reception of ultrasound waves and the image generator 112 stops the ultrasound image generation process. Subsequently, the display device 119 displays the ultrasound image generated by the display screen generator 117 immediately prior to stopping. In any other case, processing returns to step S110, and the next ultrasound image generation process is performed. In other words, an ultrasound image at a time point is generated by the processes of steps S110-S112, and this series of processes is performed in a time series with respect to a plurality of time points.

Further, storage of the ultrasound image is performed for a required time for the type classification. The required time varies depending on subject location. When the operator instructs that reproduction is to be stopped before the required time has passed, the ultrasound diagnostic device 100 does not perform the type classification and provides notification to the operator such as displaying a prompt to start again or a warning that classification will be imprecise. Further, in order to prevent mistaken instruction by the operator, the ultrasound diagnostic device 100 may display a timer bar, etc., to allow the operator to see the required time for classification and how much time has passed.

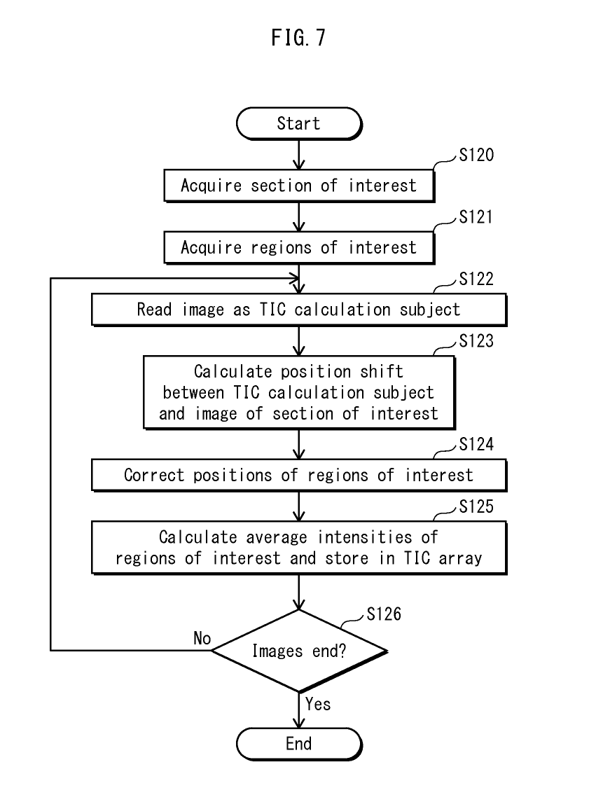

The following is a description of a TIC generation process pertaining to the present embodiment. FIG. 7 is a flowchart of the TIC generation process pertaining to the present embodiment.

The following description assumes operation after an instruction to stop reproduction in step S113 of FIG. 6. When an instruction to stop reproduction is inputted at a time before the time required to perform the type classification has passed, the ultrasound diagnostic device 100 either does not perform the type classification and prompts the operator to restart or warns the operator that results will incomplete and performs the type classification within a range of time for which reproduction has been performed. Further, the ultrasound diagnostic device 100 may automatically perform the following processing after the required time has passed even when an instruction to stop reproduction is not received.

### Step S120

When the operator instructs, via the input device 118, that type classification is to be performed, the display screen generator 117 reads the fundamental image and the harmonic image from the storage 113 and creates a display image in which the fundamental image and the harmonic image are arranged side-by-side. Further, the display screen generator 117 creates a notification for the operator, such as a message prompt to select the section of interest. The display device 119 displays the display image and the notification. The notification need not be visual information, and may be a sound, etc. For example, the notification may be speech or a sound to notify the operator. The speech or sound may be emitted from a speaker, etc., connected to the ultrasound diagnostic device body 101 or the display device 119.

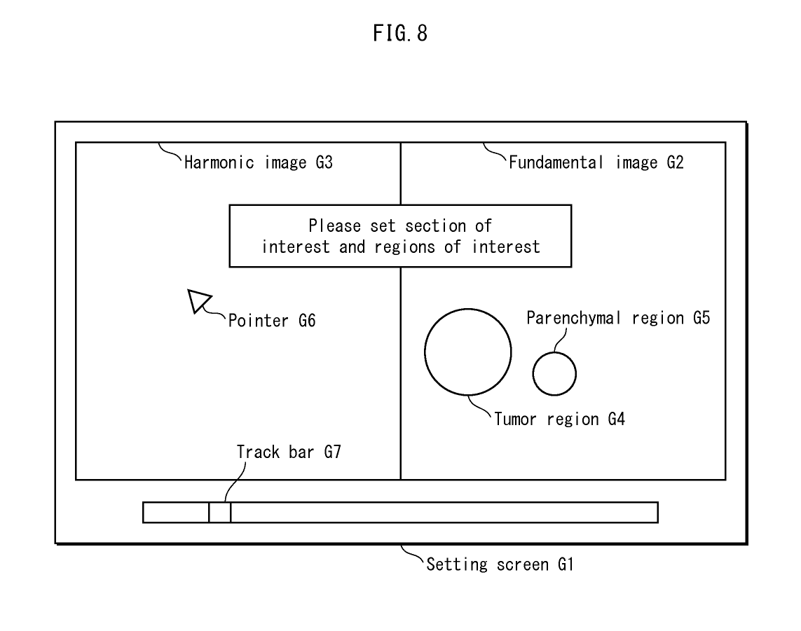

FIG. 8 is a diagram illustrating an example of a setting screen pertaining to the present embodiment. As illustrated in FIG. 8, a setting screen G1 has a fundamental image G2, a harmonic image G3, a tumor region G4, a parenchymal region G5, a pointer G6, and a track bar G7.

The operator, by using the input device 188 such as a mouse, trackball, etc., moves a pointer G6 and sets the section of interest using the track bar G7.

The input acquirer 114 stores, in the storage 113, position information of the section of interest so selected.

### Step S121

Subsequently, the display screen generator 117 generates a notification such as a message prompting selection of the regions of interest, and outputs such notification to the display device 119. Such notification may be generated at the same time as the notification prompting selection of the section of interest, as illustrated in FIG. 8, or may be generated subsequently. Further, such notification need not be visual and may be auditory.

The operator, by moving the pointer G6 using the input device 118, sets the region of interests (tumor region G4 and parenchymal region G5) with respect to the section of interest. Note that in FIG. 8, the tumor region G4 and the parenchymal region G5 are set in the fundamental image G2, but one or more of the tumor region G4 and the parenchymal region G5 may be set in the harmonic image G3.

Further, the parenchymal region G5 is preferably a region of similar size to the tumor region G4. Specifically, a position in the depth direction (a position in a horizontal direction of the ultrasound image) of the parenchymal region G5 is preferably close to a position in the depth direction of the tumor region G4.

The input acquirer 114 stores, in the storage 113, position information, etc., of the regions of interest (the tumor region G4 and the parenchymal region G5) set by the operator via the input device 118.

### Step S122

Subsequently, the motion detector 120 reads from the storage 113 a fundamental image for calculating average intensity (hereafter, “inputted fundamental image”).

### Step S123

Further, the motion detector 120 reads from the storage 113 the fundamental image of the section of interest and calculates a position shift between the fundamental image of the section of interest and the inputted fundamental image. For example, the motion detector 120 calculates the position shift by known pattern matching and detects a position shift value as a movement vector. Because the contrast agent component of the fundamental image is minor, pattern changes due to perfusion are small, and the fundamental image is suitable for motion vector detection. The motion detector 120 outputs a detected motion vector to the intensity calculator 121.

### Step S124

The intensity calculator 121 reads the regions of interest from the storage 113 and corrects the regions of interest by using the motion vector outputted by the motion detector 120. In this way, positions of the regions of interest are corrected in a plurality of images acquired in a time series.

### Step S125

The intensity calculator 121 reads a harmonic image subject from the storage 113 and calculates an average intensity of the regions of interest after position correction. Here, the regions of interest include the tumor region and the parenchymal region, and the intensity calculator 121 performs calculation of average intensity with respect to each of the two regions of interest.

The intensity calculator 121 stores, in TIC arrays of the storage 113, the average intensity of the tumor region and the average intensity of the parenchymal region.

### Step S126

The TIC generator 115 performs the above processes of step S122 to step S125 with respect to every image that is a processing subject. The TIC generator 115, when all fundamental images and harmonic images that are processing subjects are read from the storage 113, stops calculation of position shift and calculation of average intensity, and ends generation of TICs of the TIC array stored in the storage 113.

According to the above processing, a TIC of the tumor region (the tumor TIC 202) and a TIC of the parenchymal region (the parenchymal TIC 203) are generated.

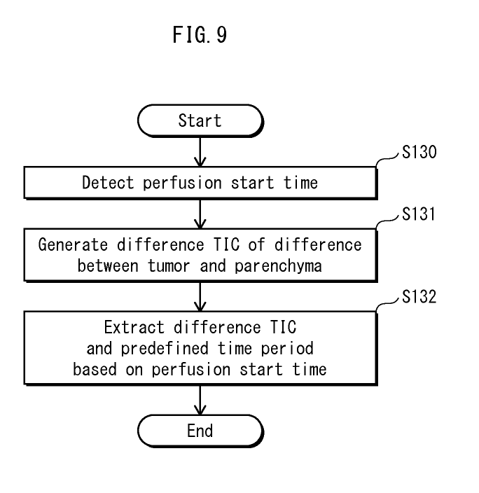

The following is a description of a TIC normalization process pertaining to the present embodiment. FIG. 9 is a flowchart of the TIC normalization process pertaining to the present embodiment.

The following description assumes an operation after the process of step S126 is finished and TIC generation is complete.

### Step S130

The perfusion time detector 130 reads the tumor TIC 202 from the storage 113 and detects a perfusion start time of the contrast agent by using the tumor TIC 202. The perfusion start time is a time at which a TIC rises. For example, a time at which the average intensity first reaches 10% of maximum intensity of the TIC. Subsequently, the perfusion time detector 130 outputs the perfusion start time to the TIC normalizer 131.

### Step S131

The TIC normalizer reads the tumor TIC 202 and the parenchymal TIC 203 from the storage 113 and generates a difference TIC that is the difference between the tumor TIC 202 and the parenchymal TIC 203. Typically, increase and decrease of a TIC is faster for a malignant tumor than for parenchymal tissue. This trend is reflected in the difference TIC.

### Step S132

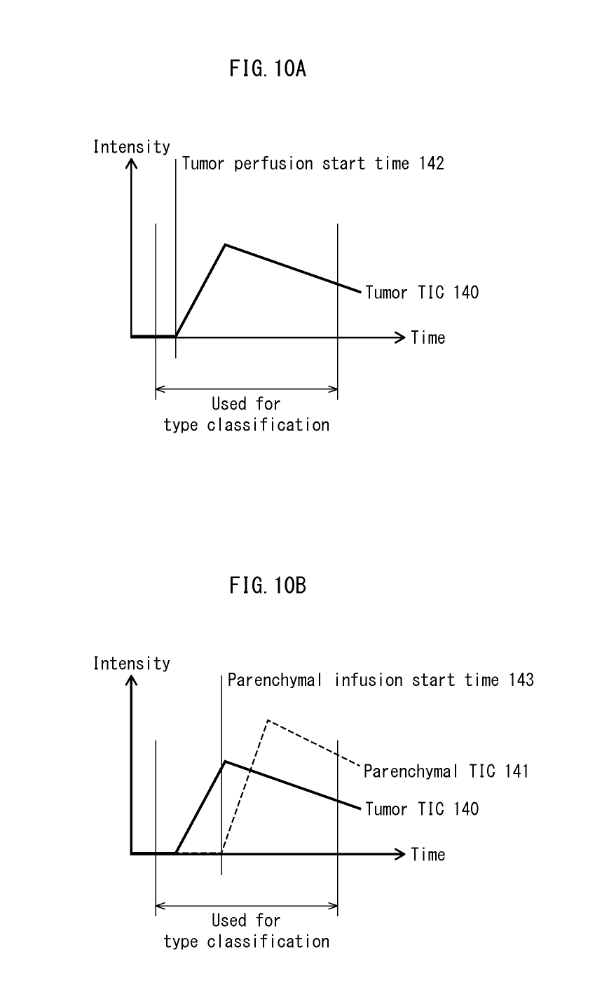

The TIC normalizer 131 resets a time of the difference TIC based on the perfusion start time detected in step S130. Further, the TIC normalizer 131 extracts a TIC used for type classification from the difference TIC after normalization. FIG. 10A is a diagram illustrating an example of a tumor perfusion start time 142, which is a perfusion start time detected from the tumor TIC 140, and a section of a TIC used in type classification with the tumor perfusion start time 142 as a reference point.

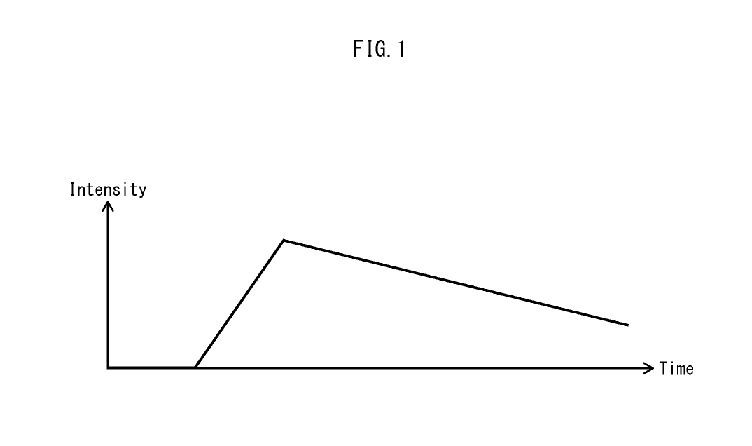

Note that, as illustrated in FIG. 10B, a parenchymal perfusion start time 143, which is a perfusion start time detected from the parenchymal TIC 141, may be used as a reference point instead of the tumor perfusion start time. FIG. 1 OB is a diagram illustrating an example of a section of the TIC used in type classification with the parenchymal perfusion start time 143 as a reference point. In such a case, the calculation of the difference TIC in step S131 need not be performed. The reason for this is that even when calculation of the difference TIC is not performed, type classification can be performed taking into account the difference between a malignant tumor and parenchymal tissue.

According to the above, a normalized TIC is generated.

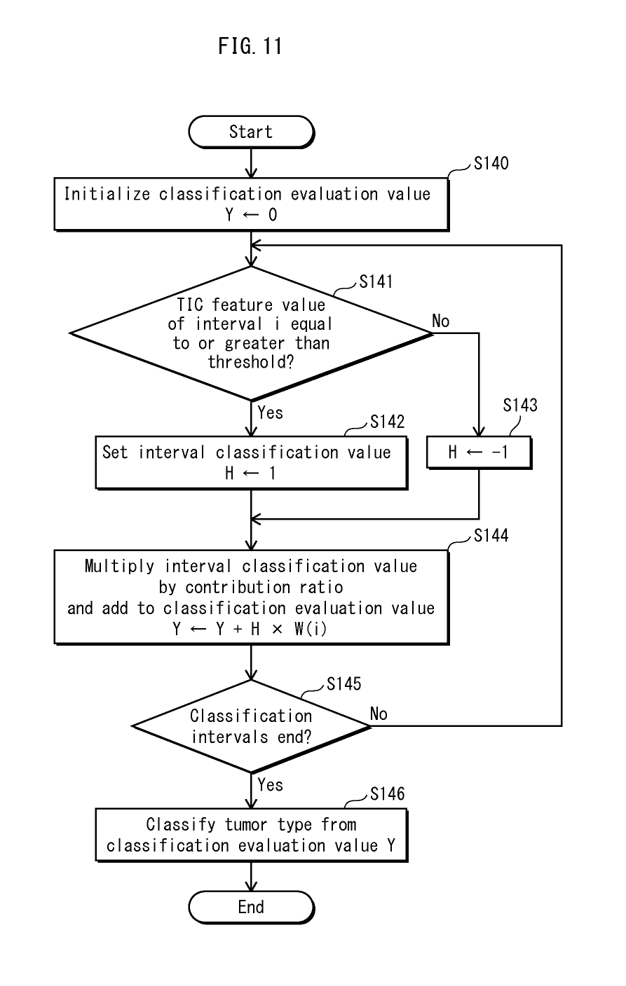

The following is a description of a type classification process pertaining to the present embodiment. FIG. 11 is a flowchart of a tumor type classification process of the present embodiment.

The following description assumes an operation after the process of step S132 is finished and TIC normalization is complete.

Step S140 First, the final classifier 134 initializes a classification evaluation value Y as zero.

### Step S141

Subsequently, the interval classifier 132 reads classification intervals and thresholds from the storage 113.

The classification intervals and the thresholds are described below.

The classification intervals are, as mentioned above, time intervals having a high contribution ratio to type classification. There is at least one classification interval, and although the number of classification intervals varies according to the living organism and the tumor, there are preferably at least two classification intervals, one either side of the perfusion start time.

The thresholds are provided one for each classification interval, and are different for each classification interval. Such thresholds are parameters used when determining how close a feature value (average intensity, variance, gradient, etc.) of a corresponding classification interval is to a given type.

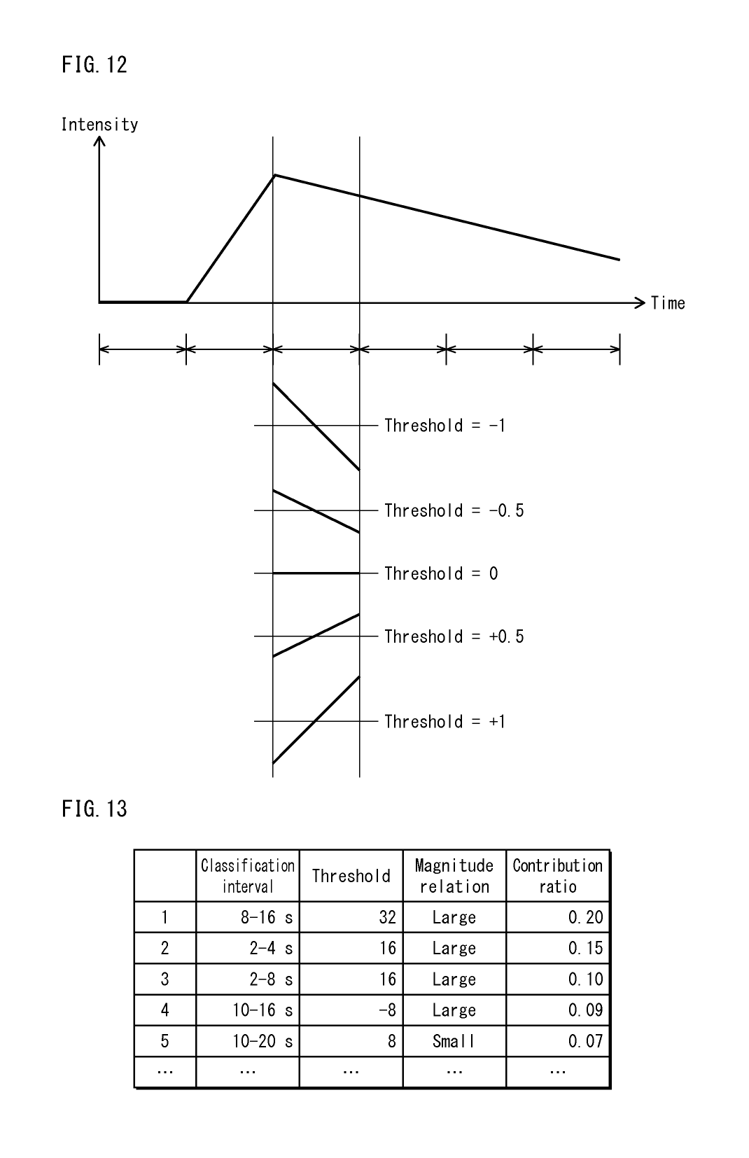

The classification intervals and the thresholds are calculated by using machine learning algorithms such as boosting. Typically, the classification intervals and the thresholds are determined by another device, and results of such classification are stored in the storage 113. In the machine learning, contribution ratios to type classification are calculated for possible combinations of time interval and threshold. FIG. 12 illustrates thresholds selected by such machine learning. The total number of combinations is, for example, when there are 100 time interval patterns, five patterns indicating thresholds as illustrated in FIG. 12, and two patterns indicating which is greater out of each pair of the feature values of classification intervals and the thresholds, a total of 1000 (100×5×2). Among such patterns, at least one pattern having a high contribution ratio is stored in the storage 113 in a format as illustrated in FIG. 13.

Note that the thresholds illustrated in FIG. 12 are examples in which gradients of average intensity are used as thresholds. When a threshold is a negative value, average intensity in the classification interval is decreasing and whether or not the gradient is equal to or greater (or less) than the threshold is determined. When a threshold is a positive value, average intensity in the classification interval is increasing and whether or not the gradient is equal to or greater (or less) than the threshold is determined.

The interval classifier 132 performs type classification with respect to an inputted TIC (the difference TIC after normalization) by comparing, for each classification interval, a TIC feature value within the classification interval and a threshold.

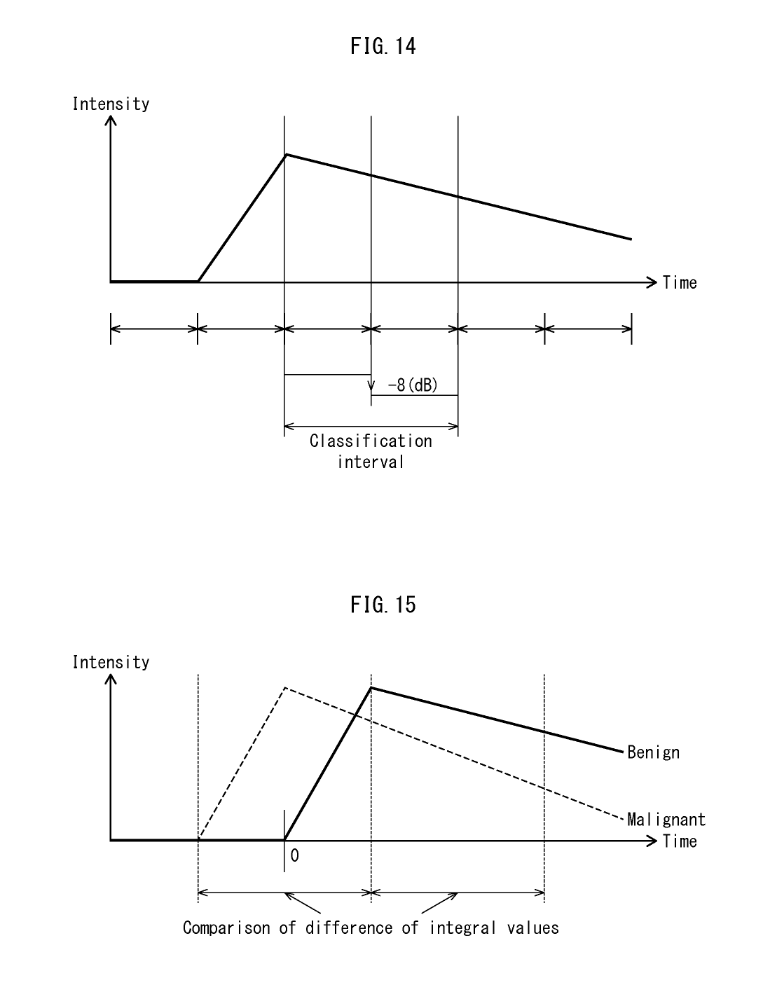

Further, the interval classifier 132 may determine whether or not a difference between an average intensity of a first half interval and a second half interval included in the classification interval is equal to or greater (or less) than the threshold, as illustrated in FIG. 14. Further, such a difference in average intensity and such a threshold may be a ratio of average intensity (for example, in decibels (dB)) of the first half interval to the second half interval. In this way, by using the ratio, appropriate classification can be performed without data indicating image capture conditions, etc.

TICs of benign tumors and malignant tumors have the following characteristics.

In a TIC of a benign tumor, “staining” timing (perfusion start time) is equivalent to that of a parenchymal TIC. Further, in a TIC of a benign tumor, staining continues for a relatively long time (intensity decreases slowly).

In a TIC of a malignant tumor, staining timing (perfusion start time) is earlier than that of a parenchymal TIC, staining does not continue (intensity decreases rapidly), and staining is poor (intensity increase is small).

Taking into account the above characteristics, the interval classifier 132 may calculate a difference of integral values of the first half interval and the second half interval, and may compare the difference and the thresholds, as illustrated in FIG. 15.

In this way, the interval classifier 132 performs type classification with respect to an inputted TIC by comparing, for each classification interval, a TIC feature value within a classification interval and a threshold. The inputted TIC may be a difference TIC that is a difference between a tumor TIC and a parenchymal TIC, and may be a tumor TIC itself. Further, the inputted TIC may be a TIC obtained by normalization of a difference TIC or a tumor TIC, and may be a difference TIC or a tumor TIC that is not normalized. Here, normalization is a process of matching time of a TIC to the reference time. The reference time is a time at which a parenchymal TIC or a tumor TIC begins increasing.

Further, the interval classifier 132 compares the intensity and the threshold of the inputted TIC within the classification interval. For example, the interval classifier 132 compares an average value of intensity included in the classification interval and a threshold. Alternatively, the interval classifier 132 compares a gradient of intensity included in the classification interval and a threshold gradient. Alternatively, the interval classifier 132 compares a difference (or ratio) of an average value or integral value of intensity between two intervals included in the classification interval and a threshold. Here, the two intervals include, for example, adjacent intervals, one of which includes the perfusion start time.

### Step S142

In the classification interval, when a TIC feature value is equal to or greater than the threshold, the interval classifier 132 sets an interval classification value H to 1.

### Step S143

On the other hand, when a TIC feature value is less than the threshold, the interval classifier 132 sets the interval classification value H to −1.

The interval classifier 132 outputs the interval classification value H to the contribution multiplier 133.

### Step S144

Subsequently, the contribution multiplier 133 reads a contribution ratio W of a machine learning parameter from the storage 113, multiplies the interval classification value H outputted by the interval classifier 132 by the contribution ratio W, and adds the multiplication result to a classification evaluation value Y. Note that the contribution ratio W is obtained by the above-mentioned machine learning.

### Step S145

The type classifier 116 performs steps S141-S144 for all classification intervals.

### Step S146

After calculation of the classification evaluation value Y is finished for all classification intervals, the final classifier 134 classifies tumor type based on the classification evaluation Y. For example, the final classifier 134 classifies that a tumor is benign when the classification evaluation value is positive and classifies that a tumor is malignant when the classification evaluation value is negative.

In the above description, average intensity of a TIC is calculated from intensity. However, for example, intensity prior to image quality adjustment by the operator may be used, and ultrasound signals (RF signals) may be used. In this way, performance dependency on operator settings is avoided.

Further, in the above description, the ultrasound diagnostic device 100 performs type classification by using a TIC indicating a time series variation of average intensity. However, instead of average intensity, other information indicating a contrast-enhanced pattern may be used. For example, variance, kurtosis, skewness, etc., may be used as such information. In this way, type classification is performed taking into account a contrast-enhanced pattern.

Further, in the above description, tumors are classified into two types, benign and malignant, but classification into three or more types is also possible. In such a case, classification into combinations of two classes is possible, for example. For example, when classifying into three classes, A, B, and C, the ultrasound diagnostic device 100 performs classification into A and C, B and C, and C and A, and a type that is selected the most is selected as the type of tumor. Further, when the number of selected classes is equal, the ultrasound diagnostic device 100 selects a type having a high classification evaluation value.

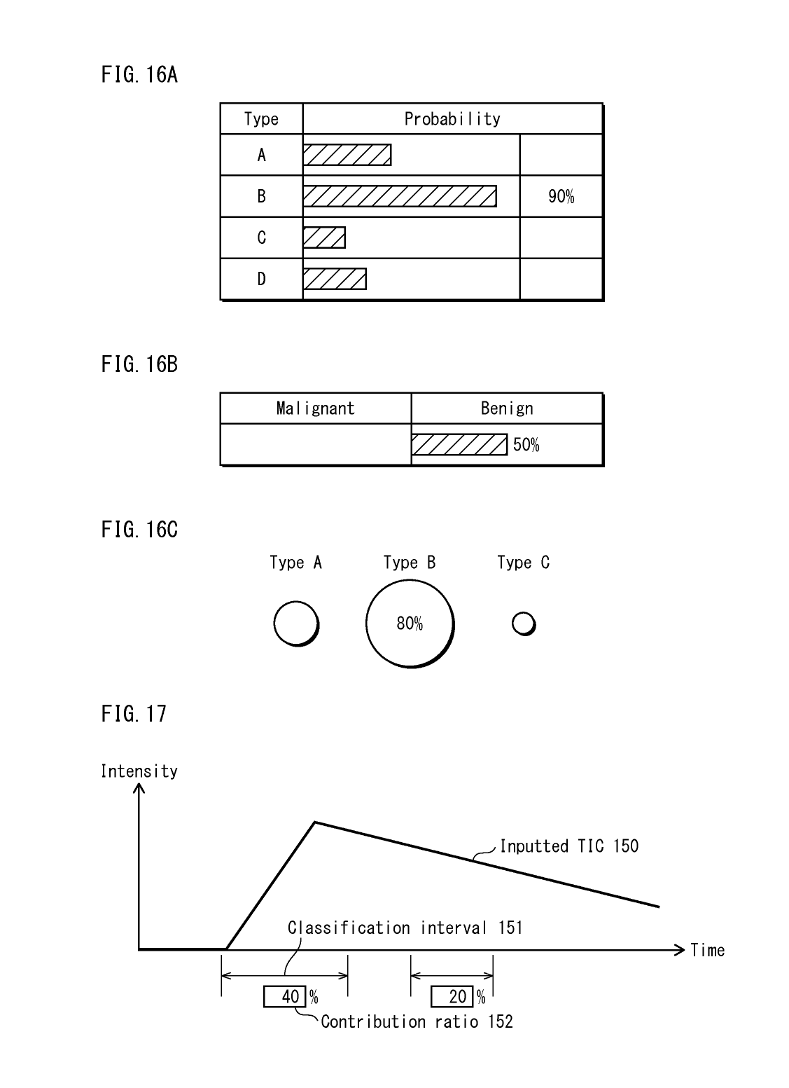

Further, the ultrasound diagnostic device 100, as a classification result, when a plurality of types exist, may (1) display one type having a highest type probability (classification evaluation value), (2) display a predefined number of types (for example, three) starting from a type having a highest type probability, or (3) display types having a type probability equal to or greater than a predefined type probability. Further, as illustrated in FIG. 16A and FIG. 16B, the ultrasound diagnostic device 100 may display type probability as bars. Further, as illustrated in FIG. 16C, the ultrasound diagnostic device 100 may represent type probability as marks of varying size. Further, the ultrasound diagnostic device 100 may emphasize the type having the highest type probability. For example, the ultrasound diagnostic device 100 may display the type having the highest type probability by changing a color, displaying text in bold, and displaying in a larger size.

Further, as illustrated in FIG. 17, the ultrasound diagnostic device 100 may display a TIC used in classification (inputted TIC 150), and may further display a classification interval 151 and a contribution ratio 152 corresponding to the classification interval 151. Further, the operator may change the contribution ratio 152 via the input device 118. When the contribution ratio 152 is changed, the ultrasound diagnostic device 100 performs type classification using the contribution ratio after the change. In this way, type classification may be performed again, based on the experience, etc., of the operator.

<Effects>

As described above, the ultrasound diagnostic device 100 pertaining to the present embodiment is capable of directly dealing with time series data of average intensity. In this way, because pre-processing such as fitting does not cause useful information to be lost in type classification, performance of type classification is improved.

Further, the ultrasound diagnostic device 100 pertaining to the present embodiment classifies type by using an interval useful for type classification that is calculated in advance by machine learning. In this way, because type is classified based around an interval useful for type classification, the ultrasound diagnostic device 100 improves performance of type classification.

Further, the ultrasound diagnostic device 100 pertaining to the present embodiment uses a difference TIC that is a difference between the tumor TIC and the parenchymal TIC in tumor type classification. In this way, because the ultrasound diagnostic device 100 takes into account a perfusion time difference between a malignant tumor and parenchymal tissue, performance of type classification is improved.

Further, the ultrasound diagnostic device 100 pertaining to the present embodiment normalizes TIC data used in tumor type classification with a perfusion start time of a parenchymal TIC as a reference. In this way, because the ultrasound diagnostic device 100 takes into account a perfusion time difference between a malignant tumor and parenchymal tissue, performance of type classification is improved.

Further, the ultrasound diagnostic device 100 pertaining to the present embodiment classifies tumor type based on changes of average intensity over a time series. Here, the average intensity is not dependent on scale. Accordingly, the ultrasound diagnostic device 100 implements type classification that is not dependent on a scaling ratio at a time of image acquisition.

### Embodiment 2

In the present embodiment, a portion of the ultrasound diagnostic device 100 corresponding to the medical data processing device is described.

<Structure>

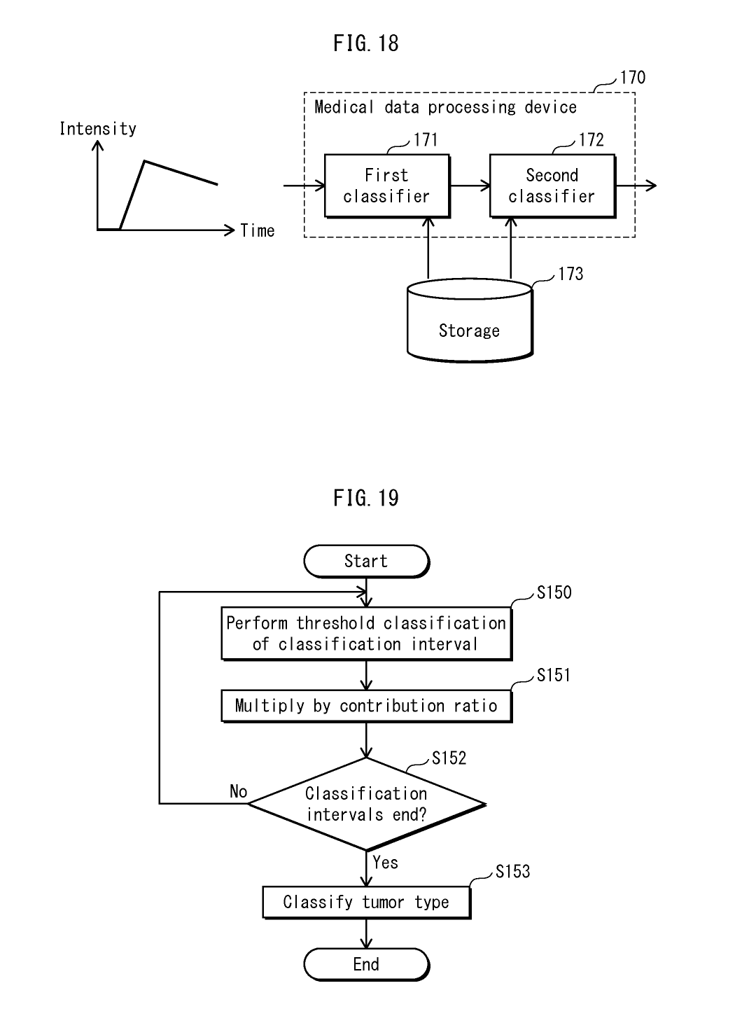

FIG. 18 is a block diagram illustrating structure of a medical data processing device 170.

As illustrated in FIG. 18, the medical data processing device 170 includes a first classifier 171 and a second classifier 172, and is connected by wired or wireless means to the storage 173. The first classifier 171 corresponds to the perfusion time detector 130, the TIC normalizer 131, the interval classifier 132, etc., pertaining to embodiment 1. The second classifier 172 corresponds to the contribution multiplier 133, the final classifier 134, etc., pertaining to embodiment 1.

The medical data processing device 170 classifies tumor type by using a first numerical sequence (TIC) indicating changes of a feature value in a time series of a tumor region including a tumor. Here, the first numerical sequence is obtained from echo signals obtained from a living organism after administration of a contrast agent, as mentioned above. The feature value may be average intensity, variance, gradient, etc. Further, the feature value may be a difference of the feature value (for example, intensity) between the tumor region including a tumor and the parenchymal region not including the tumor, and may be the feature value of the tumor region itself.

The TIC (first numerical sequence) corresponding to the tumor type classification is inputted to the first classifier 171. The first classifier 171 reads, from the storage 173, at least one set of a classification interval of the TIC used in tumor type classification and a threshold. Further, the first classifier 171 performs threshold classification for TIC feature values within the interval of each classification interval. The classification intervals and the thresholds, and classification using the classification intervals and the threshold, are the same as described in embodiment 1.

In other words, the first classifier 171 extracts, from the first numerical sequence, a first numerical sequence portion of a classification interval having a predefined time period shorter than the entire time period of the first numerical sequence and classifies the tumor type by using the first numerical sequence portion. Specifically, the first classifier 171, with respect to each classification interval previously set, classifies tumor type by using the first numerical sequence portion of the classification interval and outputs the intermediate results thereof, which indicate classification results.

For example, the first classifier 171 compares the intensity value and the threshold of the first numerical sequence portion within the classification interval. Specifically, the first classifier 171 compares the average value of intensity included in the classification interval and the threshold. In other words, the first classifier 171 classifies the tumor type based on which is greater out of an average value of the first numerical sequence portion included in the classification interval and a preset threshold.

Alternatively, the first classifier 171 may classify tumor type based on which is greater out of a time change value of the first numerical sequence portion included in the classification interval and a preset threshold. Specifically, the first classifier 171 may compare a gradient of intensity included in the classification interval and a threshold gradient. Alternatively, the first classifier 171 may compare a difference (or ratio) of an average value or integral value of intensity between two intervals included in the classification interval and the threshold.

Further, as described above, the first classifier 171 may normalize the difference TIC or the tumor TIC. Here, normalization is a process of matching time of a TIC to the reference time. The reference time is a time at which the parenchymal TIC or the tumor TIC begins increasing. In other words, the first classifier 171 may acquire a second numerical sequence (parenchymal TIC) indicating a time series variation of a feature value of a parenchymal region that does not include the tumor and determine a perfusion start time of the contrast agent from the second numerical sequence. Further, the classification interval may be a predefined time interval based on a determined perfusion start time as the reference time.

A threshold classification result for each classification interval determined is inputted to the second classifier 172. The second classifier 172 reads a contribution ratio from the storage 173, multiplies the threshold classification result by the contribution ratio corresponding to a classification interval, calculates a sum of multiplication results for all classification intervals, and classifies the tumor type by using the sum. Specifically, the second classifier 172 reads, from the storage 173, a table, etc., in which the sum of the multiplication results and tumor types are associated, and classifies tumor type by using the calculated sum and the table.

In other words, the second classifier 172 classifies the tumor type by using the intermediate results and the contribution ratios pre-associated with each classification interval. Specifically, the second classifier 172, for each classification interval, calculates a multiplication result by multiplying the intermediate result of the classification interval by the contribution ratio pre-associated with the classification interval, calculates a multiplication sum by summing all the multiplication results, and classifies the tumor type based on the multiplication sum.

Note that the first classifier 171 may use only one classification interval. In such a case, multiplication processing of the contribution ratio by the second classifier 172 does not have to be performed. In other words, the medical data processing device 170 may output the threshold classification result of one classification interval as the tumor type classification result. Here, the one classification interval is an interval that is predefined as having a high contribution ratio. Accordingly, even in such a case, performance of tumor type classification may be improved compared to use of the entirety of the first numerical sequence.

Further, a classified tumor type may be displayed on a display, etc., connected to the medical data processing device 170. In other words, the medical data processing device 170 may include a display that displays the tumor type classified by the first classifier 171 and the second classifier 172. For example, the display may correspond to the display device 119 illustrated in FIG. 2.

Further, the first classifier 171 or the second classifier 172 may determine probabilities indicating whether a tumor is one of various types of tumor. The display, as illustrated in FIGS. 16A, 16B, and 16C, may display a plurality of tumor types and probabilities of a tumor being a given tumor type. Further, the display may display such probabilities as graphics as illustrated in FIG. 16A and FIG. 16B. Further, the display may emphasize the type having the highest probability among the plurality of types.

Further, as illustrated in FIG. 17, the display may display the first numerical sequence as a graph and display, associated with the graph of the first numerical sequence, the classification intervals and the contribution ratios corresponding to the classification intervals. Further, the medical data processing device 170 may further comprise an input device that receives a change to the contribution ratios made by an operator, and the second classifier 172 may re-classify the tumor type based on the change made to the contribution ratios. Here, the input corresponds to the input device 118 illustrated in FIG. 2.

The classification intervals, thresholds, and contribution ratios described above are calculated by application of a machine learning algorithm such as boosting to TICs related to a tumor, the type of which is to be classified.

As described in embodiment 1, the classification intervals are intervals having high contribution ratios to tumor classification in the time interval group 160 illustrated in FIG. 5. The first classifier 171 classifies, for each interval, which type of tumor the feature value (average intensity, intensity change, etc.) of the TIC in an interval is close to. The thresholds are parameters used in the classifications, and are different for each interval.

When boosting is applied to the classifications, one set of the classification intervals, the thresholds, and the greater/lesser relationships is defined as a weak classifier. By machine learning, a contribution ratio of each weak classifier is determined. FIG. 13 illustrates a table for type classification that is an example of machine learning results stored in the storage 173.

Here, the classification interval may be a time period of increase or decrease where the contribution ratio is greater than a predefined value and has a large influence on type classification, and may be each interval dividing up an entire time period of a TIC. When the entire time period of a TIC is divided up, the time period may be equally divided and each interval associated with a contribution ratio, and may be divided into periods of different length of large changes such as an increase or a decrease and periods without such large changes. The classification intervals of a TIC and the thresholds and contribution ratios associated with the classification intervals are stored in storage 173.

Further, a table used in the above classification is a table such as the table illustrated in FIG. 13. Thresholds are different values according to machine learning parameters and an ultrasound device that acquires a TIC, and are not limited to the values in the table illustrated in FIG. 13. Depending on the living organism, tumor, and tumor position, adjustment of numbers and values of the thresholds is required.

A table pre-created and incorporating such adjustments is stored in the storage 173.

<Operations>

FIG. 19 is a flowchart illustrating an operation of the medical data processing device 170.

### Step S150

When a TIC to be used in classification is inputted, the first classifier 171 reads one classification interval and threshold from the storage 173 and performs threshold classification using the threshold read from the storage 173.

### Step S151

The second classifier 172 multiplies the threshold classification result by a contribution ratio corresponding to the classification interval read from the storage 173.

### Step S152

When the multiplication has not been performed for every classification interval stored in the storage 173, processing returns to step S150, and when the multiplication has been performed for every classification interval stored in the storage 173, processing proceeds to step S153. In other words, the processing of step S150 and S151 is performed for every classification interval.

### Step S153

The second classifier 172 reads a table indicating tumor types from the storage 173, and classifies tumor type from a sum Y of multiplication results that are values each multiplied by a contribution ratio of a corresponding classification interval of a TIC.

<Effects>

As described above, according to the medical data processing device 170 pertaining to the present embodiment, performance of tumor type classification is improved by using information useful in type classification extracted from a TIC.

Other Modifications

Note that the present invention has been described based on the above embodiments but the present invention is not limited to the above embodiments. The following cases are also included in the present invention.

(1) Each of the above devices is a computer system composed from a microprocessor, ROM, RAM, hard disk unit, display unit, keyboard, mouse, etc. A computer program is stored in the RAM or the hard disk unit. The microprocessor operates according to the computer program, and each device implements functions thereof. Here, the computer program is composed of a combination of a plurality of pieces of instruction code that instructs a computer to implement the predefined functions.

(2) An entire element or a portion of an element composing each of the above devices may be composed of a single system large scale integration (LSI). Such a system LSI is an ultra multi-function LSI in which multiple elements are integrated into a single chip, and is a computer system including a microprocessor, ROM, RAM, etc. A computer program is stored in the RAM. The microprocessor operates according to the computer program, and the system LSI implements functions thereof.

(3) An entire element or a portion of an element composing each of the above devices may be composed of an IC card or single module that is attachable to and detachable from a corresponding device. Such an IC card or module is a computer system composed of a microprocessor, ROM, RAM, etc. Such an IC card or module may include an ultra multi-function LSI as described above. The microprocessor operates according to the computer program, and the IC card or module implements functions thereof. Such an IC card or module may be rendered tamper resistant.

(4) The present invention may be the method indicated above. Further, the present invention may be a computer program implementing the method via a computer, and may be a digital signal composed of the computer program.

Further, the present invention may be stored as the computer program or the digital signal on a computer-readable non-transitory storage medium, for example, a flexible disk, hard disk, CD-ROM, MO, DVD, DVD-ROM, DVD-RAM, Blu-Ray Disc (registered trademark), semiconductor memory, etc. Further, the present invention may be the digital signal stored on such a storage medium.

Further, the present invention may be transmitted as the computer program or the digital signal via telecommunication lines, wireless, wired communication lines, networks such as the interne, data broadcasts, etc.

Further, the present invention may be a computer system including a microprocessor and memory, the memory storing the computer program and the microprocessor operating according to the computer program.

Further, the program or the digital signal may be implemented by an independent computer system by storage and transfer by the storage medium or by transmission via the network, etc.

(5) The above embodiments and the above modifications may be combined.

Further, in each of the above embodiments, each element may be composed of specialized hardware or may be implemented by execution of a software program suitable for each element. Each element may be implemented by a program executor such as a CPU or processor reading a software program stored on a storage medium such as a hard disk or semiconductor memory and executing the software program.

Further, values used above are all examples used to describe the present invention in detail, and the present invention is not limited to the values used as examples.

Further, division into functional blocks in the block diagrams represent examples. Multiple functional blocks may be implemented as a single functional block, one functional block may be divided into multiple functional blocks, and portions of a function may be moved to another functional block. Further, functions of a plurality of functional blocks having similar functions may be processed by a single piece of hardware or software processing in parallel or in time division.

Further, processes implementing steps included in the above processing are examples used to describe the present invention, and may be processes other than those described above. Further, a portion of the above steps may be implemented at the same time (in parallel) as another step.

Above, description is provided based on embodiments of an ultrasound diagnostic device and medical data processing device pertaining to one or multiple functions but the present invention is not limited to the embodiments. Without departing from the spirit of the present invention, various modifications that occur to those skilled in the art may be applied to the embodiments and even a form constructed by combining elements into a different embodiment may include one or more functions.

### INDUSTRIAL APPLICABILITY

The present invention is applicable to ultrasound diagnostic devices. Further, the present invention may be used in qualitative diagnosis by ultrasound waves using a contrast agent.

### REFERENCE SIGNS LIST

100 ultrasound diagnostic device
101 ultrasound diagnostic device body
110 ultrasound probe
111 ultrasound transmitter-receiver
112 image generator
113 storage
114 input acquirer
115 TIC generator
116 type classifier
117 display screen generator
118 input device
119 display device
120 motion detector
121 intensity calculator
130 perfusion time detector
131 TIC normalizer
132 interval classifier
133 contribution multiplier
134 final classifier
140 tumor TIC
141 parenchymal TIC
142 tumor perfusion start time
143 parenchymal perfusion start time
150 inputted TIC
151 classification interval
152 contribution ratio
160 time interval group
170 medical data processing device
171 first classifier
172 second classifier
173 storage
200 cine image
201 regions of interest
202 tumor TIC
203 parenchymal TIC
204 classification interval
205 classification threshold
206 contribution ratio
207 classification result
208 machine learning data
G1 setting screen
G2 fundamental image
G3 harmonic image
G4 tumor region
G5 parenchymal region
G6 pointer
G7 track bar

1. A medical data processing device that classifies a tumor type by using a first numerical sequence indicating a time series variation of a feature value of a tumor region including a tumor, the first numerical sequence being obtained from echo signals obtained from a living organism after administration of a contrast agent, the medical data processing device comprising:
a first classifier that extracts, from the first numerical sequence, a first numerical sequence portion of a classification interval having a predefined time period shorter than the entire time period of the first numerical sequence and classifies the tumor type by using the first numerical sequence portion.

2. The medical data processing device of claim 1, wherein:
the classification interval is provided in a plurality,
the first classifier classifies the tumor type by using the first numerical sequence portion of each classification interval and outputs a plurality of corresponding intermediate results, which each indicate a result of the first classifier classifying the tumor type, and
the medical data processing device further comprises a second classifier that classifies the tumor type by using the intermediate results and contribution ratios pre-associated with the classification intervals on a one-to-one basis.

3. The medical data processing device of claim 2, wherein the second classifier, for each classification interval, calculates a multiplication result by multiplying the intermediate result of the classification interval by the contribution ratio pre-associated with the classification interval, calculates a multiplication sum by summing all the multiplication results, and classifies the tumor type based on the multiplication sum.

4. The medical data processing device of claim 1, wherein the feature value is a difference between intensity of the tumor region and a parenchymal region that does not include the tumor.

5. The medical data processing device of claim 1, wherein:
the first classifier acquires a second numerical sequence indicating a time series variation of a feature value of a parenchymal region that does not include the tumor and classifies a perfusion start time of the contrast agent from the second numerical sequence, and
the classification interval is a time interval predefined by using the perfusion start time as a reference time.

6. The medical data processing device of claim 1, wherein the first classifier classifies the tumor type based on which is greater out of an average value of the first numerical sequence portion included in the classification interval and a preset threshold.

7. The medical data processing device of claim 1, wherein the first classifier classifies the tumor type based on which is greater out of a time change value of the first numerical sequence portion included in the classification interval and a preset threshold.

8. The medical data processing device of claim 1, further comprising:
a display that displays the tumor type as classified by the first classifier.

9. The medical data processing device of claim 8, wherein:
the first classifier classifies probabilities of the tumor being each of a plurality of types, and
the display displays the plurality of types and the probabilities of the tumor being each of the types.

10. The medical data processing device of claim 9, wherein the display displays the probabilities as a graphic.

11. The medical data processing device of claim 9, wherein the display emphasizes a highest probability type among the plurality of types.

12. The medical data processing device of claim 2, further comprising:
a display that displays the first numerical sequence as a graph and displays, associated with the graph of the first numerical sequence, the classification intervals and the contribution ratios corresponding to the classification intervals.

13. The medical data processing device of claim 12, further comprising:
an input device that receives a change to the contribution ratios made by an operator, wherein the second classifier re-classifies the tumor type based on the change made to the contribution ratios.

14. A medical data processing method of classifying a tumor type by using a first numerical sequence indicating a time series variation of a feature value of a tumor region including a tumor, the first numerical sequence being obtained from echo signals obtained from a living organism after administration of a contrast agent, the medical data processing method comprising:
a first classifying step of extracting, from the first numerical sequence, a first numerical sequence portion of a classification interval having a predefined time period shorter than the entire time of the first numerical sequence and classifies the tumor type by using the first numerical sequence portion.

15. A program causing a computer to execute the medical data processing method of claim 14.

16. An ultrasound diagnostic device, comprising:
an ultrasound probe that acquires echo signals from a living organism after administration of a contrast agent;
a numerical sequence generator that generates, from the echo signals, a first numerical sequence that indicates a time series variation of a feature value of a tumor region including a tumor; and
the medical data processing device of any one of claim 1 that classifies tumor type by using the first numerical sequence.
